(19) United States
(12) Patent Application Publication
(10) Pub. No.: US 2004/0229873 A1
(43) Pub. Date: Nov. 18, 2004

(54) Treatment of neurodegenerative conditions

(75) Inventors: Laurence S. Harbige, London (GB); Michael J. Leach, West Wickham (GB); Mohammed Sharief, London (GB)

Correspondence Address: NIXON & VANDERHYE, PC, 1100 N GLEBE ROAD, 8TH FLOOR, ARLINGTON, VA, 22201-4714, US

(73) Assignee: BTG International Limited, London (GB)

(21) Appl. No.: 10/756,761

(22) Filed: Jan. 14, 2004

(30) Foreign Application Priority Data
Jan. 14, 2003 (GB) 0300783.8

(51) Int. Cl. A61K 31/53; A61K 31/505; A61K 31/50; A61K 31/44

(52) U.S. Cl. 514/242; 514/247; 514/275; 514/352

(57) ABSTRACT

A method of treating a patient in need of therapy for multiple sclerosis is provided, comprising administering to that patient a therapeutically effective dose of a compound of formula I

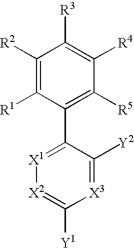

during periods of remission, as well as during relapse, wherein R¹, R², R³, R⁴ and R⁵ are independently selected from the group consisting of hydrogen, trihaloalkyl and halo substituents; X¹, X² and X³ are independently selected from the group consisting of CH, CCH₂F, CCF₃, COalkyl and CCH₃, and nitrogen atoms, with at two of X¹, X² and X³ being nitrogen; and Y¹ and Y² are independently selected from the group consisting of hydrogen and primary, secondary and tertiary amino groups.

Preferred compounds of formula 1 is selected from the group consisting of Lamotrigine, Sipatrigine, 4030w92, 202w92, 78c90 (active Sipatrigine metabolite), 440c89, 149C89, 722c90, 279c90 and 1003c87.

The therapy results in reduction of one or more of incidence of relapse, spasticity and fatigue and exceptionally the therapy stabilises the patients Expanded Disability Status Score (EDSS), thus halting progress of the disease.

The present invention relates to a method for treating multiple sclerosis, particularly remitting/relapsing multiple sclerosis, using the particular class of selected sodium channel blocking compounds including Lamotrigine and Sipatrigine.

Multiple sclerosis is an inflammatory and demyelinating disease of the CNS the cause of which is unknown. Strong evidence indicates that progress involves immune mediated mechanisms (Brosnan & Raine (1996): Noseworthy (1999)). The processes of axonal damage in MS, particularly, chronic inflammation, demyelination and astrogliosis is complex, but white matter inflammation and demyelination are considered to determine disease severity. Recent studies (De Stefano et al (2001)) have also suggested that axonal damage begins in the early stages of the disease and contributes to disability. It is a highly complex disease and can be conversely exacerbated and ameliorated by the activity of T-cells and other immune response factors.

Essential fatty acid containing oils have been shown to be effective in the EAE animal model of multiple sclerosis, with some having been shown to have an effect in reducing duration and severity of symptoms in man in vivo. Other treatments, such as use of cyclosporin, are shown to be effective in the EAE model, as with oils, but where these are employed in the human multiple sclerosis disease, whilst symptoms improve, the underlying disease continues to progress. The ‘gold standard’ treatment for MS remains interferon, such as with β-Avonex®, Rebif® and other interferon preparations. This gold standard treatment only addresses needs of some of the patients and even in these symptom improvement is restricted. Typically a proportion, eg. about 30% of patients, have their number of relapses reduced in incidence by about a third.

The symptoms of MS are classified as either positive or negative; positive symptoms include painful tonic seizures, itching, acute and chronic pain and negative symptoms including paralysis. Positive symptoms are presumed to result from abnormal high frequency impulses generated at the sites of demyelination and negative symptoms from blockade of nervous conduction.

An endogenous peptide sodium channel blocking factor which has local anaesthetic properties has been identified in the cerebral spinal fluid of patients with multiple sclerosis (Brinkmeier et al 2000; Aulkemeyer et al 2000) and it has been suggested that this factor may actually contribute to the conduction block and partial paralysis seen in MS. However, low doses of local anaesthetics such as lidocaine given by iv infusion, and its analogue mexiletine, generally improve clinical positive symptoms rather than worsening negative symptoms (Sakurai and Kanazawa 1999). Lidocaine has a voltage and frequency dependent block of sodium channels with preferential blockade of high frequency firing. Following their clinical evaluation of the local anaesthetics, Sakurai and Kanazawa concluded that compounds which preferentially block high frequency abnormal impulses with no effect on transmission have a potential utility in MS.

WO 99/52522 relates to the use of sodium channel blockers for the treatment of neurological disorders associated with inflammation of the central or peripheral nervous system, including inter alia multiple sclerosis, for which it is particularly suggested that they might be useful when the patient is in a relapse state. Drugs suggested for this use include sodium channel blockers lignocaine, carbamazepine, phenytoin and lamotrigine.

WO 98/46574 and WO 00/61231 similarly suggest use of sodium channel blockers for alleviating pain and other effects of neurodegenerative diseases such as MS. Surprisingly, in the light of such teachings, sodium channel bl0ckers such as carbamazepine and phenytoin have subsequently been used to treat symtoms of MS (see Sakurai and Kanazawa 1999) without appropriate success to justify their replacement of existing drug therapy. In fact carbamazapene has been found to seriously enhance disability in multiple sclerosis patients even at relatively low doses (Ramsaransing et al (2000) BMJ Vol 320, p 1113 22 Apr. 2000).

Lamotrigine is a sodium channel blocking compound which prolongs the inactivation state of the sodium channel as well as having voltage and use dependent actions. Both Lamotrigine and a related sodium channel blocker Sipatrigine protect white matter against ischemic injury as well as grey matter (Garthwaite et al (1999)) where studies in rat optic nerve in vitro evaluated the degree of neuroprotection afforded to white matter axons by drug following oxygen and glucose deprivation and provided dramatic (up to 90%) dose related neuroprotection of white matter. The concentrations of Sipatrigine, a compound with a distinct activity profile to Lamotrigine, are in the same range as brain concentrations achieved following a single bolus dose of sipatrigine in rat pMCAO.

The present inventors have now surprisingly determined that compounds of the Lamotrigine family, far from being ineffective or damaging in MS, are far more effective than even the gold standard interferons in its treatment. Unlike the other sodium channel blockers, they do not involve unwanted side effects yet prove exceptionally efficacious in reducing incidence of relapse, reducing spasticity and reducing fatigue. Furthermore, use of these compounds stabilises the patients Expanded Disability Status Score (EDSS), thus indicating that disease progress is halted. Particularly beneficial is the ability of these drugs to reduce relapse rate when administered prophylactically rather than when such incidences occur.

In order to produce such effect the inventors have employed increased dosage over that usually employed for the known anti-epileptic indication for these compounds, and particularly dose escalate from a sub-optimal therapeutic dose to a supra-optimal therapeutic dose for such epileptic indication.

In a first aspect of the present invention there is provided a method of treating a patient in need of therapy for multiple sclerosis comprising administering to that patient a therapeutically effective dose of a compound of formula I

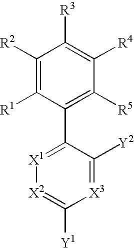

wherein R¹, R², R³, R⁴ and R⁵ are independently selected from the group consisting of hydrogen, trihaloalkyl and halo substituents;

X¹, X² and X³ are independently selected from the group consisting of CH, CCH₂F, CCF₃, COalkyl and CCH₃, and nitrogen atoms, with at two of X¹, X² and X³ being nitrogen, alkyl being preferably ethyl, ethyl or propyl; and Y¹ and Y² are independently selected from the group consisting of hydrogen and primary, secondary and tertiary amino groups.

Preferably R1 to R5 are independently selected from hydrogen and chloro, with two or three of R1 to R5 being chloro.

In a first preferred embodiment X1, X2 and X3 are nitrogen, in a second preferred embodiment X1 is selected from the group consisting of CH and CCH₂F and X² and X³ are nitrogen and in a third preferred embodiment X1 and X3 are nitrogen and X² is CH.

Preferably Y1 is selected from —NH₂, -1-piperazinyl and 4-alkyl-1-piperazinyl and Y2 is —NH², alkyl being preferably methyl, ethyl or propyl.

Most preferred compounds of formula 1 are selected from the group consisting of Lamotrigine: 3,5-diamino-6-(2,3-dichlorophenyl)-1,2,4-triazine, Sipatrigine: 4-amino-2-(4-methyl-1-piperazinyl)-5-(2,3,5-trichlorophenyl)-pyrimidine, 2,4-diamino-5-(2,3-dichlorophenyl)-6-(fluoromethylpyrimidine), R-(−)-2,4-diamino-6-fluoromethyl-5-(2,3,5-trichlorophenyl)-pyrimidine, 4-amino-2-(1-piperazinyl)-5-(2,3,5-trichlorophenyl)-pyrimidine (active Sipatrigine metabolite), 4-amino-2-(4-methyl-1-piperazinyl)-5-(2,3,5-trichlorophenyl)-6-trifluoromethylpyrimidine, 2,4-diamino-5-(2,3,5-trichlorophenyl)-trifluoromethylpyrimidine, 2,4-diamino-5-(2,3,5-trichlorophenyl)-6-methoxymethylpyrimidine, 4-amino-6-methyl-2-(4-methyl-1-piperazinyl)-5-(2,3,5-trichlorophenyl)-pyrimidine, 4-amino-2-(4-propyl-1-piperazinyl)-5-(2,3,5-trichlorophenyl)-pyrimidine and 2,4-diamino-5-(2,3,5-trichlorophenyl)-pyrimidine. Several of these compounds are described in U.S. Pat. Nos. 5,635,507, 5,597,828, 5,684,005, 5,587,380, 5,712,276 and 5,712,277 all of which are incorporated herein by reference.

Preferably, and most surprisingly, the therapy results in reduction of one or more of incidence of relapse, degree of spasticity and fatigue, particularly daytime fatigue. More preferably the therapy stabilises the patients Expanded Disability Status Score (EDSS), thus halting progress of the disease. The EDSS is conveniently that described by Kurtzke, Neurology, (1983), 33:1444-52, incorporated herein by reference.

Preferably the compound of formula 1 is administered during periods of remission, as well as during relapse, such that the occurrence of relapse is reduced.

Preferably the compound of formula I is given at a dose sufficient to reduce spasticity or daytime fatigue. Dosing is conveniently orally administered by tablet or capsule, but may be by any conventional dosing route.

Preferably the compound is administered at a dose of from 400 mg/day to 1000 mg/day, more preferably about 500 mg/day to 700 mg/day and most preferably 600 mg/day. More preferably the compound is administered in an escalating dosing regime, starting at 100 mg/day or less and escalating to the maximum treatment dose over a period of 1 to 10 weeks, more preferably over 3 to 6 weeks.

In demonstrating the therapy of the present invention, Lamotrigine was given to seven patients suffering from MS to provide symptomatic therapy for epileptic seizures, for which it has been licensed, or chronic pain of neurological origin, for which it is a recognised treatment. The average maintenance dose of Lamotrigine in these conditions is 400 mgs per day, but the initial dose or the rate of escalation may cause several side effects.

In the present study, the inventors followed the clinician inventor's clinical practice, which adopted two strategies that had varied from the manufacturer's recommendations. Firstly, a very slow titration process starting with 25 mgs per day increasing the dose gradually (often over several weeks) to avoid adverse events. Secondly, a maintenance dose with mean of 600 mgs per day was applied. Using these two strategies, the inventors observed improvement in the clinical activity of MS and also stabilisation of the disease process. The beneficial effects are illustrated in the following two case studies compared with matching patients who had not been treated with Lamotrigine.

### EXAMPLE 1

1a. Lamotrigine Treatment

The first treated patient was a woman aged 36 years. She had a relapsing remitting MS for four years, and had an average of one severe clinical relapse per year. She was referred to the clinician inventor in 1998, and her clinically active disease at that time represented a valid indication for treatment with diseases modifying therapy (β-interferon or Copaxone). However, the therapy could not be started because of financial constraints that were then imposed by her Health Authority. She also complained of sharp pain affecting the legs, which was resistant to several types of analgesia and also resistant to tricyclic antidepressants. Accordingly, treatment was started with Lamotrigine to treat her pain.

The initial dose was 25 mgs per day, which was escalated gradually as outlined above. Her Expanded Disability Status Scale at that time was 2.5. Her painful symptoms improved following Lamotrigine therapy. She also noticed improvement in her other MS-related symptoms, such as muscle spasticity in the legs and daytime fatigue. Moreover, there was a significant improvement in her MS disease activity in that she had no clinical relapses since 1998. Her EDSS score remained stable at 2.5. Such improvement seemed to be related to Lamotrigine therapy because she had received no immunosuppressive or steroid therapy during the past four years.

1b Control Treatment.

This improvement contrasts with that of another woman with active relapsing remitting MS (aged 37 years) who had MS for 3½ years before developing intractable pain. She also had annual clinical relapses, and was treated with tricyclic antidepressant (Amitriptyline), which improved her painful symptoms. However, her MS remained active, and she continued to have clinical relapses on a yearly basis.

### EXAMPLE 2

2a Lamotrigine Treatment.

A second of Lamotrigine-treated patient was a man aged 23 years, who was diagnosed with relapsing remitting MS five years ago. He also had idiopathic generalised epilepsy since childhood, which was treated with sodium valproate. In the first two years following the diagnosis of MS, he had three clinical relapses. His seizures also deteriorated and because of this, his anti-epileptic therapy was changed from sodium valproate to Lamotrigine. This drug was introduced at a low dose, and was escalated cautiously as outlined above. His EDSS score at the beginning of treatment with Lamotrigine was 1.5. Since such treatment, his MS disease activity improved significantly (no clinical relapses) and his EDSS remained stable at 1.5. This improvement seemed to be related to Lamotrigine therapy since he had received no treatment with steroids or immunosuppressive drugs.

2b Control Treatment

In contrast, another man of a similar age who was also diagnosed with relapsing remitting MS five years ago, was still experiencing annual clinical relapses because of lack of specific therapy. His EDSS score at the time of diagnosis was 1.5, but his condition has now deteriorated and his current EDSS score is 5.5.

### EXAMPLES 3-7

Five other patients also treated with Lamotrigine were also experienced clinical improvement in their MS activity following treatment illustrating that the drug exerts a novel immunomodulatory effect in this disease.

1. A method of treating a patient in need of therapy for multiple sclerosis comprising administering to that patient a therapeutically effective dose of a compound of formula I 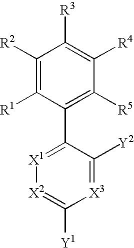
wherein R¹, R², R³, R⁴ and R⁵ are independently selected from the group consisting of hydrogen, trihaloalkyl and halo substituents;
X¹, X² and X³ are independently selected from the group consisting of CH, CCH₂F, CCF₃, COalkyl and CCH₃, and nitrogen atoms, with at two of X¹, X² and X³ being nitrogen, alkyl being preferably ethyl, ethyl or propyl; and Y¹ and Y² are independently selected from the group consisting of hydrogen and primary, secondary and tertiary amino groups.

2. A method as claimed in claim 1 wherein R¹ to R⁵ are independently selected from hydrogen and chloro, with two or three of R¹ to R⁵ being chloro.

3. A method as claimed in claim 1 wherein X¹, X² and X3 are nitrogen.

4. A method as claimed in claim 1 wherein X¹ is selected from the group consisting of CH and CCH₂F and X² and X³ are nitrogen.

5. A method as claimed in claim 1 wherein X¹ and X³ are nitrogen and X² is CH.

6. A method as claimed in claim 1 wherein Y¹ is selected from —NH₂, -1-piperazinyl and 4-alkyl-1-piperazinyl and Y2 is —NH₂.

7. A method as claimed in claim 1 wherein the compound of formula 1 is selected from the group consisting of Lamotrigine: 3,5-diamino-6-(2,3-dichlorophenyl)-1,2,4-triazine, Sipatrigine: 4-amino-2-(4-methyl-1-piperazinyl)-5-(2,3,5-trichlorophenyl)-pyrimidine, 2,4-diamino-5-(2,3-dichlorophenyl)-6-(fluoromethylpyrimidine), R-(−)-2,4-diamino-6-(fluoromethyl)-5-(2,3,5-trichlorophenyl)-pyrimidine, 4-amino-2-(1-piperazinyl)-5-(2,3,5-trichlorophenyl)-pyrimidine (active Sipatrigine metabolite), 4-amino-2-(4-methyl-1-piperazinyl)-5-(2,3,5-trichlorophenyl)-6-trifluoromethylpyrimidine, 2,4-diamino-5-(2,3,5-trichlorophenyl)-trifluoromethylpyrimidine, 2,4-diamino-5-(2,3,5-trichlorophenyl)-6-methoxymethylpyrimidine, 4-amino-6-methyl-2-(4-methyl-1-piperazinyl)-5-(2,3,5-trichlorophenyl)-pyrimidine, 4-amino-2-(4-propyl-1-piperazinyl)-5-(2,3,5-trichlorophenyl)-pyrimidine and 2,4-diamino-5-(2,3,5-trichlorophenyl)-pyrimidine.

8. A method as claimed in claim 1 wherein the therapy results in reduction of one or more of incidence of relapse, spasticity and fatigue.

9. A method as claimed in claim 1 wherein the therapy stabilises the patients Expanded Disability Status Score (EDSS), thus halting progress of the disease.

10. A method as claimed in claim 1 wherein the compound of formula 1 is administered during periods of remission, as well as during relapse, such that the occurrence of relapse is reduced.

11. A method as claimed in claim 1 wherein the compound of formula I is given at a dose sufficient to reduce spasticity or daytime fatigue.

12. A method as claimed in claim 1 wherein the compound of formula 1 is administered at a dose of from 400 mg/day to 1000 mg/day.

13. A method as claimed in claim 1 wherein the compound of formula 1 is administered at a dose of 500 mg/day to 700 mg/day.

14. A method as claimed in claim 1 wherein the compound of formula 1 is administered at a dose of about 600 mg/day.

15. A method as claimed in claim 1 wherein the compound is administered in an escalating dosing regime, starting at 100 mg/day or less and escalating to the maximum treatment dose over a period of 1 to 10 weeks.
